(19) United States
(12) Patent Application Publication
(10) Pub. No.: US 2011/0212189 A1
(43) Pub. Date: Sep. 1, 2011

(54) GASTRORETENTIVE ORAL HIGH DOSE ZINC PREPARATIONS

(75) Inventors: George J. BREWER, Ann Arbor, MI (US); Steve H. KANZER, Ann Arbor, MI (US); David A. NEWSOME, Saint Petersburg, FL (US)

(73) Assignee: ADEONA PHARMACEUTICALS, INC., Ann Arbor, MI (US)

(21) Appl. No.: 12/917,231

(22) Filed: Nov. 1, 2010

Related U.S. Application Data
(60) Provisional application No. 61/257,034, filed on Nov. 1, 2009.

(51) Int. Cl. A61K 33/30; A61K 33/00; C07F 3/06; A61P 1/00

(52) U.S. Cl. 424/643; 424/717; 548/101

(57) ABSTRACT

A sustained-release zinc composition preferably includes potassium bicarbonate. A method of providing zinc to a subject in need of treatment includes administering to the subject an effective amount of a sustained-release zinc composition which preferably includes potassium bicarbonate.

### CROSS-REFERENCE TO RELATED APPLICATIONS

Priority of our U.S. Provisional Patent Application Ser. No. 61/257,034, filed 1 Nov. 2009, incorporated herein by reference, is hereby claimed.

Incorporated herein by reference are all patents and patent applications naming one or more of us as inventors, or naming Adeona as assignee, including:

U.S. patent application Ser. No. 11/621,962, filed 10 Jan. 2007, titled PHARMACEUTICAL COMPOSITIONS AND METHODS TO ACHIEVE AND MAINTAIN A TARGETED AND STABLE COPPER STATUS AND PREVENT AND TREAT COPPER-RELATED CENTRAL NERVOUS SYSTEM DISEASES, published as US 2007-0207191 A1 on 6 Sep. 2007, which claims priority to U.S. Provisional Patent Application Ser. No. 60/765,812, filed 7 Feb. 2006, titled PHARMACEUTICAL COMPOSITIONS AND METHODS TO ACHIEVE AND MAINTAIN A TARGETED AND STABLE COPPER STATUS AND PREVENT AND TREAT COPPER-RELATED CENTRAL NERVOUS SYSTEM DISEASES and to U.S. Provisional Patent Application Ser. No. 60/757,672, filed 10 Jan. 2006, titled PHARMACEUTICAL COMPOSITIONS AND METHODS TO ACHIEVE AND MAINTAIN A TARGETED COPPER STATUS;

PCT Patent Application No. PCT/US2007/60345, filed 10 Jan. 2007, published as WO 2007/084818 A2, which claims priority to U.S. Provisional Patent Application Ser. No. 60/765,812, filed 7 Feb. 2006, titled PHARMACEUTICAL COMPOSITIONS AND METHODS TO ACHIEVE AND MAINTAIN A TARGETED AND STABLE COPPER STATUS AND PREVENT AND TREAT COPPER-RELATED CENTRAL NERVOUS SYSTEM DISEASES and to U.S. Provisional Patent Application Ser. No. 60/757,672, filed 10 Jan. 2006, titled PHARMACEUTICAL COMPOSITIONS AND METHODS TO ACHIEVE AND MAINTAIN A TARGETED COPPER STATUS;

U.S. Provisional Patent Application 60/894,388 filed 12 Mar. 2007, titled ORAL ZINC MEDICANTS USEFUL FOR SAFELY LOWERING FREE COPPER ABSORPTION AND FREE COPPER LEVELS and utility patent application PCT/US2008/056743 filed 12 Mar. 2008 titled ORAL ZINC MEDICANTS USEFUL FOR SAFELY LOWERING FREE COPPER ABSORPTION AND FREE COPPER LEVELS;

U.S. Provisional Patent Application Ser. No. 61/169,684, filed 15 Apr. 2009, titled METHODS AND DEVICES FOR MEASURING DEFECTIVE CERULOPLASMIN.

### STATEMENT REGARDING FEDERALLY SPONSORED RESEARCH OR DEVELOPMENT

Not applicable

### REFERENCE TO A “MICROFICHE APPENDIX”

Not applicable.

### BACKGROUND OF THE INVENTION

1. Field of the Invention

The present invention relates to nutritional supplements and/or pharmaceutical agents. More particularly, the present invention relates to nutritional supplements or pharmaceutical agents providing zinc to a subject in need of treatment.

2. General Background of the Invention

Zinc, an essential nutrient, is the second most abundant trace element in the human body and the most abundant trace element in the eye. It is necessary for the activity of more than 200 enzymes and for the DNA binding capacity of over 400 nuclear regulatory elements. There is evidence that zinc may function as an antioxidant by protecting sulfhydryl groups from oxidation, competing with copper and iron to reduce the formation of hydroxyl radicals which are a result of redox cycling and by the induction of the antioxidant protein metallothionein (MT) which can scavenge damaging hydroxyls.

It has been suggested that oxidative stress and a decrease in antioxidant capacity play a role in several pathological conditions such as cardiovascular disease, carcinogenesis, neurological disorders, inflammatory, central nervous system, respiratory, renal diseases, reperfusion injury, and macular degeneration. Age-related macular degeneration (AMD) is the number one cause of blindness in people over 60 in the United States. It is thought that it is an age-related defect in the retinal pigment epithelium (RPE) which contributes to this disease, however, the etiology is unknown and currently there is no cure. Our laboratory has previously reported that the antioxidants catalase, MT, and zinc decrease with age and signs of age-related macular degeneration in isolated human retinal pigment epithelial cells.

Zinc has been implicated in beneficial effects on certain prostate conditions and functions, immune system function, and cancer.

Cysteine is a non-essential amino acid necessary for the formation of sulfur containing compounds such as pyruvate, taurine, and glutathione, important in normal tissue metabolism protection and repair. The presence of cysteine in proteins is generally thought to impart a protective function including antioxidant activity.

Synthesis of glutathione is largely regulated by cysteine availability. An increase in glutathione levels are beneficial when the body encounters toxic conditions such as peroxide formation, ionizing radiation, alkylating agents, or other reactive intermediates.

In premature infants, cysteine levels are low, thereby making them more susceptible to oxidative damage of hydroperoxides formed in the eye after hyperbaric oxygen treatments.

As demonstrated by Uzzo et al., zinc can inhibit the development and progression of prostate cancer. Uzzo, R. G. et al., “Zinc inhibits nuclear factor-kappa B activation and sensitizes prostate cancer cells to cytotoxic agents” Clin. Cancer Res. 2002 8:3579-83.

Zinc has been demonstrated to be clinically beneficial in many dermatological conditions. For example, see the multicenter randomized study of Pierard C. Franchiment et al., “A Multicenter Randomized Trial of Ketoconazole 2% and Zinc Pyrythione 1% Shampoos in Severe Dandruff and Seborrheic Dermatitis”, Skin Pharmacol Appl Skin Physiol. 2002 15:434-41.

The following US patents, and all references mentioned herein and in U.S. Pat. Nos. 6,586,611 and 7,164,035, are incorporated herein by reference:
U.S. Pat. No. 5,844,089 Genetically Fused Globin-like Polypeptides Having Hemoglobin-like Activity
U.S. Pat. No. 5,844,088 Hemoglobin-like Protein Comprising Genetically Fused Globin-like Polypeptides
U.S. Pat. No. 5,801,019 DNA Encoding Fused Alpha-beta Globin Pseuodimer and Production of Pseudotetrameric Hemoglobin
U.S. Pat. No. 5,798,227 Co-expression of Alpha and Beta Globins
U.S. Pat. No. 5,744,329 DNA Encoding Fused Di-beta Globins and Production of Pseudotetrameric Hemoglobin
U.S. Pat. No. 5,739,011 DNA for the Production of Multimeric Hemoglobins
U.S. Pat. No. 5,599,907 Production and Use of Multimeric Hemoglobins
U.S. Pat. No. 5,545,727 DNA Encoding Fused Di-alpha Globins and Production of Pseudotetrameric Hemoglobin
U.S. Pat. No. 5,401,770 Antipruritic Agents and Antipruritic Compositions Thereof
U.S. Pat. No. 3,941,818 1:1 Zinc Methionine Complexes
U.S. Pat. No. 4,021,569 Method of Nutritional Supplementation For Zinc and Methionine by Ingesting 1:1 Zinc Methionine Complexes
U.S. Pat. No. 4,764,633 Ferric ion Catalyzed Complexation of Zinc and/or Manganese With Alpha Amino Acids
U.S. Pat. No. 6,429,219 Treatment of Chronic Hypertension and Related Conditions with Thiol Complexes
U.S. Pat. No. 6,586,611 Zinc-monocysteine Complex and Method of Using Zinc-cysteine Complexes
U.S. Pat. No. 7,164,035 Zinc-monocysteine Complex and Method of Using Zinc-cysteine Complexes
U.S. Pat. No. 6,531,608 Various Thial Complexes, Processes For Their Synthesis and Clinical Applications
U.S. Pat. No. 5,911,978 Hair Treatment Composition
WO2007/084818 Pharmaceutical Compositions and Methods to Achieve and Maintain a Targeted and Stable Copper Status and Prevent and Treat Copper-related Central Nervous System Diseases

U.S. Pat. No. 5,401,770 discloses the use of a zinc-cysteine complex in an external use antipruritic agent.

AREDS utilized 69.6 mg/day zinc. The AREDS formula patent (U.S. Pat. No. 6,660,297) does not cover rapid improvement in visual acuity however.

Tanaka et al., “The Inhibitory Effect And The Mechanism Of Ethanol Absorption By Zinc Complex In Mouse Gastrointestinal Tract”, Folia Pharmacological Society Japan 111, 327-336 (1998);

Zegzhda et al., “COMPLEX COMPOUNDS OF ZINC AND CADMIUM WITH CYSTEIN”, COORDINATION CHEMISTRY, Vol. 2, No. 8, pp. 1031-1035 (1976).

### BRIEF SUMMARY OF THE INVENTION

The present invention includes an embodiment of a composition and method disclosed in Patent Publication No. US 2007/0207191 A1, especially in paragraphs 92 through 141, paragraph 158, and claims 15, 30, 24, 25, and 36. Patent Publication No. US 2007/0207191 A1 is incorporated herein by reference.

The assignee has both developed and exclusively in-licensed a portfolio of issued and pending US and international patent applications covering oral zinc preparations for the treatment of Alzheimer's disease, Mild Cognitive Impairment, Age-Related Macular Degeneration, Wilson's disease, Schizophrenia, Huntington's disease, Lou Gehrig's disease (ALS) and other neurodegenerative disorders. Two of the co-inventors of the current application including early provisional and utility applications related hereto, David A. Newsome, M.D. and George J. Brewer, M.D. have pioneered the use of oral zinc therapy for neurodegenerative diseases. Newsome is the inventor and pioneer of oral zinc therapy for age-related macular degeneration (now standard of care for the dry form of AMD) and George J. Brewer, M.D., is the inventor and pioneer of oral zinc acetate therapy for Wilsons' disease (now standard of care and FDA approved as Galzin®). Building upon the decades of experience of these two clinical researchers whom are intimately familiar with the significant shortcomings of existing oral zinc therapy preparations, the assignee, as described in previous related patent applications and the current patent application has invented and reduced to practice a technologically advanced oral zinc preparation with far superior tolerability and dosing convenience compared to prior and currently marketed zinc preparations. The approach is unique and the advantages are considerable.

a. Improved Tolerability.

Oral high dose zinc preparations are associated with a high incidence of dose dependent gastric irritation which typically manifests as nausea and abdominal pain. As a result, oral zinc preparations, such as those commonly used for AMD, are generally recommended to be taken with food, as the oral zinc will bind foodstuffs in the stomach and thereby reduce the potential for gastric irritation. However, numerous studies conducted by Dr. Brewer in Wilson's disease patients have shown that in order to induce intestinal metallothionein and thus have any effect in lowering non-ceruloplasmin bound (free) copper levels in serum and in turn exposure and levels of copper in the CSF/CNS compartment, oral zinc therapy must be given away from food. The oral zinc acetate capsule preparation developed by George J. Brewer, M.D. approved by the FDA in 1997 for Wilson's disease causes nausea in 45% of patients at the lowest approved dose of 25 mg elemental zinc and nausea in 91% of patients at the alternate higher approved dose of 50 mg elemental zinc. At a dose of 100 mg elemental zinc 91% of patients also experienced nausea with 18% of patients experienced vomiting. See for example, Henderson L M, Brewer G J, Dressman J B, Swidan S Z, DuRoss D J, Adair C H, Barnett J L, Berardi R R., Use of zinc tolerance test and 24-hour urinary zinc content to assess oral zinc absorption, J Am Coll Nutr. 1996 February; 15(1):79-83 and Henderson L M, Brewer G J, Dressman J B, Swidan S Z, DuRoss D J, Adair C H, Barnett J L, Berardi R R., Effect of intragastric pH on the absorption of oral zinc acetate and zinc oxide in young healthy volunteers, JPEN J Parenter Enteral Nutr. 1995 September-October; 19(5):393-7. The same effects have been observed with zinc sulphate providing 50 mg of elemental zinc in this case taken with food. Samman S, Roberts, D C K, Med. Journal Australia, 1987 Vol. 146, p. 247-9.

In the case of oral zinc therapy, the issue of gastric irritation could not be overcome merely through the use of sustained release and delayed release technology alone, since delaying the release of zinc in such fashion causes reduced bioavailability, the bypass or ineffective exposure of critical proximal intestinal sections where zinc and copper are predominately absorbed and where intestinal metallothionein required to be induced in order to affect endogenous levels of serum copper, especially non-ceruloplasmin-bound, or so-called “serum free copper.

Based upon a 90 subject prospective, observational clinical trial of 30 age-matched controls, 30 Alzheimer's disease patients and Parkinson's disease patients sponsored by the assignee, the inventors have discovered that Alzheimer's patients suffer from a subclinical and clinical zinc deficiency compared to age matched normal subjects (p=0.0145), impaired serum ceruloplasmin binding of serum copper (p=0.0000015) and increased serum percentages of non-ceruloplasmin bound copper P=0.045). The inventors have also discovered that Parkinson's disease patients also demonstrate elevated percentages serum free copper compared to normal subjects (p=0.045). Such findings were the subject of a provisional patent application 61/169,684 filed Apr. 15, 2009 entitled, “Methods and Devices for Measuring Defective Ceruloplasmin”.

The inventors have discovered that by applying gastro-retentive sustained release technology to this long standing problem, it has been able to greatly increase tolerability of oral zinc therapy without sacrificing bioavailability, minimum threshold intestinal zinc exposure required to induce metallothionein nor desired location of gastrointestinal metallothionein induction in the proximal intestines where the majority of copper is absorbed. Importantly, the achievement of such prolonged stomach retention time and delayed zinc release is accomplished entirely with excipients and binding agents (that combine the properties of pill swelling and effervescence effect in gastric juice to increase pill buoyancy for increased residence time and pill motility in the stomach). In a preferred embodiment such effect can be accomplished with 100% of ingredients and excipients all of which have Generally Regarded as Safe (GRAS) status and that are commonly used in the food industry. Further, the inventors have discovered that through the addition of basic ingredients or antacids, such as potassium bicarbonate and sodium bicarbonate, the tolerability of oral zinc therapy taken away from food can also greatly improved. Utilizing such techniques, the inventors have discovered that substantially greater oral unit doses of elemental zinc can be tolerated without nausea or gastric irritation. For example, in bioavailability studies performed in healthy subjects of appropriately formulated gastroretentive sustained release tablets of zinc acetate and zinc sulphate, 150 mg of elemental zinc are easily tolerated by subjects taken away from food for up to four weeks without any complaints of nausea or gastric irritation.

Results of studies in normal healthy human volunteers indicate that individual doses of 150 elemental zinc taken with water away from food are easily well tolerated without any observable instances of nausea or gastric irritation to date while also providing immediately observable sustained elevated serum zinc levels lasting over six hours.

b. Improved Convenience

Oral zinc therapies used for Wilson's disease and AMD both also suffer from poor patient compliance due to dosing inconvenience. In the case of Wilson's disease, oral zinc therapy must be taken away from food requiring patient dosing at least 1 hour before and 2 hours after meals. This regimen is further complicated by the fact that since most patients cannot tolerate 50 mg elemental zinc (b.i.d.) away from food due to nausea and gastric irritation, most Wilson's patients prefer to take 25 mg elemental zinc (t.i.d.) away from food. Such three times daily and narrow dosing window combine to make a regimen that is extremely difficult to comply with, as evidenced by the reported 90% incidence of nausea and gastrointestinal pain and estimated 30% non-compliance rate in this most serious copper sensitive population. In the case of AMD, the typical daily 67 mg of elemental zinc is also required to be given in divided doses taken twice per day, albeit generally with food, resulting in poor compliance due to the need for b.i.d. dosing in this older patient population already generally burdened with other concominant medication regimens. Based upon the serum zinc levels achieved in normal volunteers that have tested the inventors' gastro-retentive sustained release zinc formulation technology, the inventors believe that it has achieved the long sought after goal of once-a-day dosing for oral zinc therapy which should translate into greater patient compliance.

In a preferred embodiment, the inventors have discovered that the use of non-cellulose-based swelling/sustained release agents such as Carbopol 971P NF Polymer, Lubrizol, Cleveland, Ohio and/or Kollidon VA64, BASF, Mutchler Inc. Harrington Park N.J. provide improved zinc bioavailability compared to cellulose-based agents such HPMC, Ethyl Cellulose and Hypromellose as the latter appear to bind zinc and reduce systemic absorption and bioavailability in humans compared with non-cellulose based agents.

In a preferred embodiment the oral zinc formulation also contains the amino acid, cystiene, in order to improve bioavailability of elemental zinc, provide an amino acid source for the production of intestinal and systemic metallothionein as well as glutathione.

In a preferred embodiment, the oral zinc formulation contains an effervescent agent, such as potassium bicarbonate or sodium bicarbonate to promote gastric flotation and gastroretention of the preparation, to provide motility to the zinc preparation in the stomach thus avoiding local irritation to the stomach wall where released zinc may come in contact.

In a preferred embodiment, the oral zinc preparation contains a basic ingredient, such as potassium bicarbonate or sodium bicarbonate to reduce the local acid environment of where zinc is released in the stomach or proximal gastrointestinal system thereby reducing the potential for local irritation from the released zinc.

In a preferred embodiment, the zinc preparation contains an electrolyte such as sodium, potassium or calcium to improve bioavailability of zinc via passive and active transport mechanisms via the intestinal epithelial cell electrolyte channels.

In a preferred embodiment, the oral zinc preparation releases multiple independent gastroretentive subunits, such as microspheres, granules or particles so as to reduce the variability of the gastroretentive effect.

In a preferred embodiment, the oral zinc preparation contains an acid, such as citric acid, effervescent agent such as citric acid, stearic acid, ascorbic acid, acetic acid or zinc salts such as zinc acetate in order to facilitate the effervescent effect independent of stomach pH, to induce more rapid and more dramatic efferverscence and faster release of the zinc contained in the pill or tablet.

In a preferred embodiment, the oral zinc preparation contains zinc carnosine, in order to promote the retention of zinc via the stomach wall.

In a preferred embodiment the oral zinc preparation achieves in floating an in vitro simulated acidic gastric environment in less than 8 minutes, more preferably less than 3 minutes and more preferably under 1 minute.

In a preferred embodiment, the oral zinc preparation comprises a tablet with a rapidly dissolving microcoated layer of a sugar, polymer or other coating so as to avoid premature efferverscence in the mouth, to mask the taste of the zinc tablet and improve stability.

In a preferred embodiment, the oral zinc preparation releases 25-100 mg of elemental zinc during the first two hours of dissolution, while the preparation is retained.

In a preferred embodiment the effervescing zinc tablets are packed under an inert gas and/or in individually sealed blisters, pouches or low unit size container systems to improve stability and prevent premature effervescence.

In a preferred embodiment, the oral zinc preparation provides for immediate release of elemental zinc in the proximal gastrointestinal system during the gastroretentive phase as well as sustained release zinc for over 8 hours to deliver zinc to the small intestine and maintain bioavailable zinc for an extended period.

In a preferred embodiment, the oral zinc preparation contains over 100 mg of elemental zinc, more preferably at least 150 mg of elemental zinc.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements and wherein:

FIG. 1 shows the in vitro dissolution of Formula 1 containing 150 mg elemental zinc (as zinc acetate) utilizing a Varian VK 7010/7500/8000 dissolution testing machine utilizing a basket systems and standard ICH dissolution parameters of temperature and pH with zinc levels measured utilizing a Buck Scientific 210 VGP atomic absorption spectrophotometer.

FIG. 2 shows the average serum zinc levels achieved by ingestion of a single tablet of Formula 1 containing 150 mg elemental zinc (as zinc acetate) with serum draws up to 6 hours from serum four subjects taken in the morning away from food with a 16 oz. glass of water at least 2 hours after eating and at least 1 hour before eating with zinc levels measured utilizing a Buck Scientific 210 VGP atomic absorption spectrophotometer.

FIG. 3 shows the comparison of zinc levels achieved by ingestion of a single tablet of Formula 1 containing 150 mg elemental zinc (as zinc acetate) with serum draws up to 6 hours from serum four subjects taken in the morning away from food with a 16 oz. glass of water at least 2 hours after eating and at least 1 hour before eating with zinc levels compared to a commercially available zinc gluconate tablet providing 100 mg of elemental zinc (GNC Zinc 100) measured utilizing a Buck Scientific 210 VGP atomic absorption spectrophotometer. Note the superior are under the curve and sustained serum zinc levels achieved at 6 hours compared to the non-gastroretentive formulation which also contains cellulose. 50% of the patients taking the GNC Zinc 100 tablet away from food experienced nausea versus none of the subjects taking the tablet for Formula 1 for up to 4 weeks.

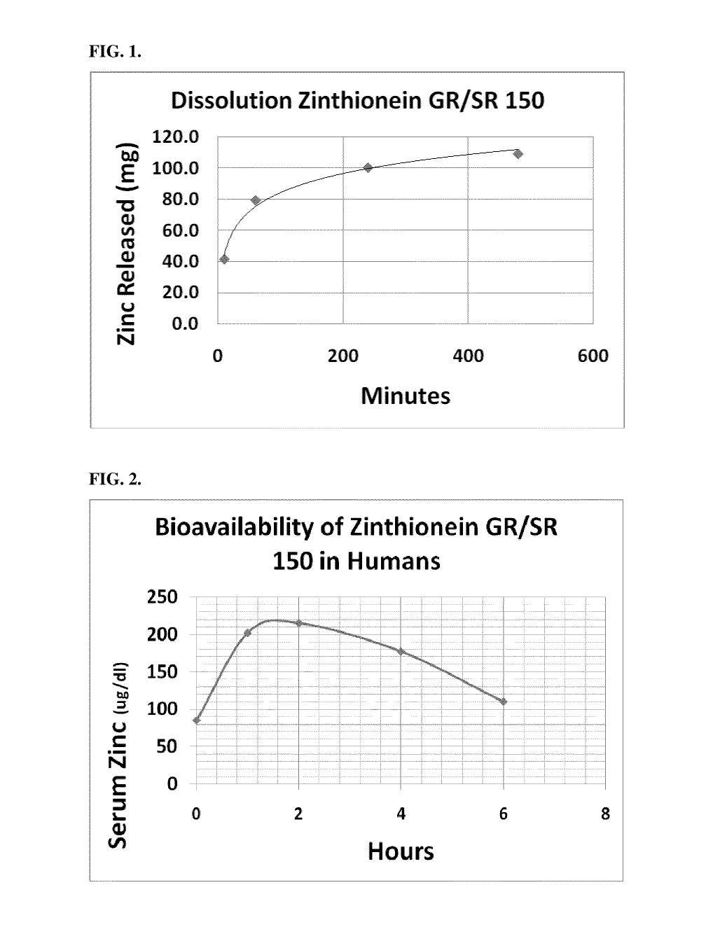

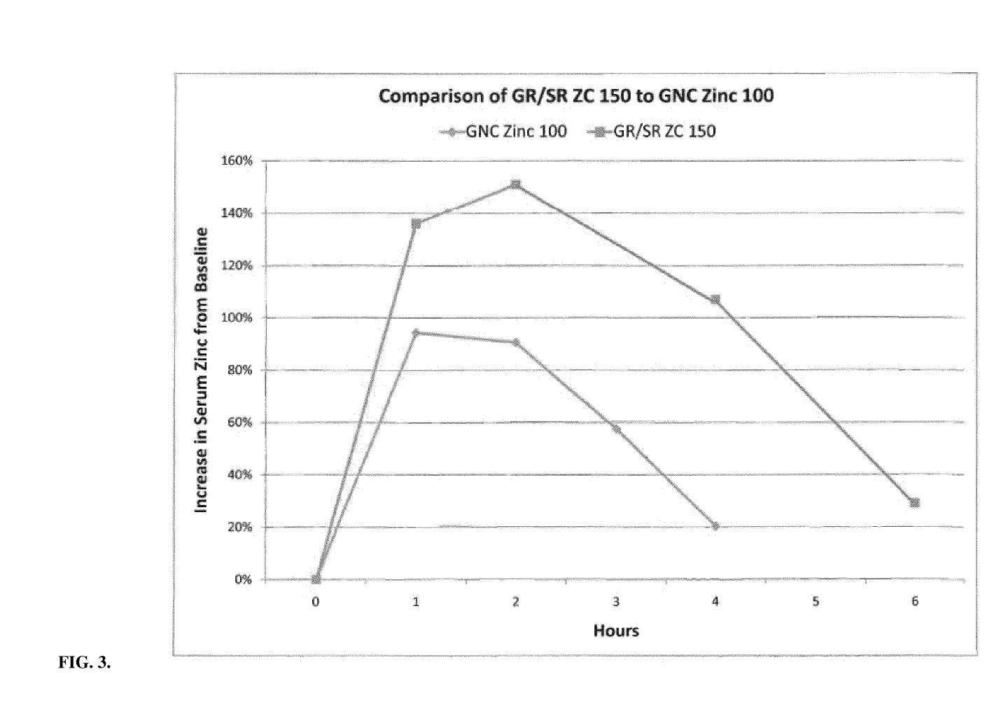

### DETAILED DESCRIPTION OF THE INVENTION

One could test the efficacy of the compositions of the present invention by using the methodology of Uzzo et al., but instead of zinc, subjects would be administered the compositions herein consistent with amounts used in published studies, including Uzzo et al.

Using randomized techniques similar to Franchiment et al., we would expect an improved clinical result with the compositions herein.

The following table indicates amounts of each ingredient per pill, when the pill is intended to be taken once daily, twice daily or three times daily:

TABLE 1
Formulations

| | Column 1 | Column 2 | Column 3 | Column 4 | Column 5 | Column 6 | Column 7 | Column 8 |
|---|---|---|---|---|---|---|---|---|
| Row | Ingredient | Function | Preferred | More | Most | Example | Example | Example |
| | | (alternatives) | amount | Preferred | Preferred | amount | amount | amount |
| | | | per pill | amount | amount | per pill | per pill | per pill |
| | | | | per pill | per pill | for one | for two | three times |
| | | | | | | pills daily | pills | daily |
| | | | | | | As used | daily | |
| | | | | | | herein | | |
| | | | | | | defined as | | |
| | | | | | | “FORM- | | |
| | | | | | | ULA 1” | | |
| 1 | zinc acetate | copper | 24 mg-1G | 80 mg-504 mg | 252 mg-504 mg | 504 mg | 252 mg | 84-168 mg |
| | | scavenger | (pro- | (provides | (provides | (provides | (provides | |
| | | (zinc | (provides | 15-150 mg | 75 mg-150 mg | 150 mg | 75 mg | |
| | | compositions | 7.5-300 mg | elemental | elemental | bio- | bio- | |
| | | providing a | elemental | zinc) | zinc) | available | available | |
| | | like amount | zinc) | | | elemental | elemental | |
| | | of elemental | | | | zinc) | zinc) | |
| | | zinc, such as | | | | | | |
| | | zinc | | | | | | |
| | | monocysteine, | | | | | | |
| | | zinc sul- | | | | | | |
| | | sulphate, zinc | | | | | | |
| | | carbonate, | | | | | | |
| | | zinc picolinate, | | | | | | |
| | | zinc carnosine, | | | | | | |
| | | zinc | | | | | | |
| | | monomethio- | | | | | | |
| | | nine, zinc | | | | | | |
| | | chloride, | | | | | | |
| | | zinc citrate, | | | | | | |
| | | zinc gluconate | | | | | | |
| | | and various | | | | | | |
| | | other zinc | | | | | | |
| | | salts, etc. | | | | | | |
| 2 | L-cysteine, | (cysteine, | 0 mg-1G | 50 mg-300 mg | 75-250 mg | 100 mg | 50 mg | 0-50 mg |
| | HCl | histidine, | | | | | | |
| | | methionine, | | | | | | |
| | | arginine) | | | | | | |
| 3 | Carbopol | swelling and | 0-300 | 50-150 | Not more | 90 mg | 100 mg | 0-95 mg |
| | 971P brand | sustained | | | than 30% of | | | |
| | polyacrylic | release agent | | | tablet weight | | | |
| | acid cros- | (Carbopol | | | | | | |
| | slinked with | 71G brand | | | | | | |
| | allyl penta | polyacrylic | | | | | | |
| | erythritol and | acid | | | | | | |
| | polymerized | crosslinked | | | | | | |
| | in ethyl acetate | with allyl | | | | | | |
| | | penta | | | | | | |
| | | erythritol and | | | | | | |
| | | polymerized in | | | | | | |
| | | ethyl acetate, | | | | | | |
| | | HPMC, | | | | | | |
| | | Kollidon | | | | | | |
| | | SR, etc.) | | | | | | |
| 4 | potassium | pH increaser | 0-300 mg | 10-250 mg | 100-200 mg | 150 mg | 120 mg | 0-150 mg |
| | bicarbonate | (antacid), | | | dependin | | | |
| | (preferably | efferves- | | | on desired | | | |
| | heat treated | vescence | | | release | | | |
| | to reduce | agent | | | curve | | | |
| | premature | (sodium | | | | | | |
| | effervescence) | bicarbonate, | | | | | | |
| | | other | | | | | | |
| | | bicarbonates | | | | | | |
| | | and bases | | | | | | |
| 5 | citric acid | Efferves- | 0-30 mg | 5-25 mg | 5-15 mg | 10 mg | 10 mg | 10 mg |
| | | vescence | | | depending | | | |
| | | promoter, for | | | on desired | | | |
| | | bioavailabili- | | | release | | | |
| | | ty(stearic | | | curve | | | |
| | | acid, ascorbic | | | | | | |
| | | acid, acitic | | | | | | |
| | | acid, acid | | | | | | |
| | | salts of zinc, | | | | | | |
| | | etc. | | | | | | |
| 6 | stearic | Lubricant, | 0-30 mg | 2-25 mg | 5-15 mg | 9 mg | 6 mg | 6-9 mg |
| | acid | acid, | | | | | | |
| | | magnesium | | | | | | |
| | | stearate, etc. | | | | | | |
| 7 | Total | | | | | 853 mg | 538 mg | |

### Examples

Tablets of Formula 1 above were made by blending 504 mg per tablet of zinc acetate dehydrate crystal USP CAS 5970-45-6, Spectrum Chemicals Inc., New Brunswick, N.J., 100 mg of L-cysteine HCL monohydrate USP, CAS 9004-57-3, Spectrum Chemicals, Inc., 90 mg Carbopol 971 P NF Polymer, Lubrizol, Cleveland, Ohio, 150 mg potassium bicarbonate granular USP, CAS144-55-8, Spectrum Chemicals Inc., 10 mg of citric acid and 9 mg of stearic acid, KIC Chemicals NF Kosher, Armonk, N.Y. Tablets were pressed on a TDP-1 benchtop single tablet press as well as a Minhua Pharmaceutical Machinery Company Co. Ltd. 40 kn 12 mm capacity rotary tablet press each utilizing an 11 mm round die set. Floating lag time and floating time of the tablets were evaluated by dropping them into a solution of water and acetic acid at a pH of 2.0. All of the tablets had floating lag time of 30 seconds to 1 minute. Dissolution testing of the tablets was tested utilizing a Varian VK 7010/7500/8000 dissolution testing system utilizing a basket systems and standard ICH dissolution parameters of temperature and pH with zinc levels measured utilizing a Buck Scientific 210 VGP atomic absorption spectrophotometer. The results demonstrate that approximately 80 mg of elemental zinc is released from the tablet over the first hour which is the inventors intended desired amount and rate of release of zinc over the period of time approximating the proximal gastrointestinal retention time followed by a slower release of the remaining zinc over a sustained period of over 8 hours approximating the sustained release in the small intestines in humans.

Single and repeated dose human bioavailability studies were conducted in 4 subjects taking 1 tablet daily for 4 weeks. FIG. 2 shows the average serum zinc levels achieved by ingestion of a single tablet of Formula 1 containing 150 mg elemental zinc (as zinc acetate) with serum draws up to 6 hours from serum four subjects taken in the morning away from food with a 16 oz. glass of water at least 2 hours after eating and at least 1 hour before eating with zinc levels measured utilizing a Buck Scientific 210 VGP atomic absorption spectrophotometer. No instances of nausea or abdominal irritation or cramping have been observed with Formula 1 in any subject taken daily away from food for 4 weeks. The significant 150 mg elemental zinc as zinc acetate dosage size so fully tolerated stands in stark contrast to the reported 90% incidence of nausea and 18% incidence of vomiting experienced by subjects taking 50 mg elemental zinc as zinc acetate in the form of the marketed zinc preparation, Galzin capsules. See for example, Henderson L M, Brewer G J, Dressman J B, Swidan S Z, DuRoss D J, Adair C H, Barnett J L, Berardi R R, Use of zinc tolerance test and 24-hour urinary zinc content to assess oral zinc absorption, J Am Coll Nutr. 1996 February; 15(1):79-83 and Henderson L M, Brewer G J, Dressman J B, Swidan S Z, DuRoss D J, Adair C H, Barnett J L, Berardi R R., Effect of intragastric pH on the absorption of oral zinc acetate and zinc oxide in young healthy volunteers, JPEN J Parenter Enteral Nutr. 1995 September-October; 19(5):393-7. The level and sustained nature of the levels of serum zinc provided by Formula 1 are also greater than those reported reported in such studies. The inventors believe that the improved tolerability of Formula 1 will allow once a day dosing away from food of 150 mg elemental zinc and indeed potentially higher single dosages since no adverse effects have been noted at 150 mg. Because oral zinc preparations intended to be given away from food has heretofore been limited to 50 mg due to tolerability issues, it is required to be given three times daily which is a major inconvenience for patients and result in poor patient compliance. The inventors consider the present invention and results to be a major advance achievement for oral zinc therapy intended to be given away from food. Such poor tolerability and inconvenience of the current preparations and prior art would greatly limit the potential utility of oral high dose zinc therapy for the general population, especially the elderly, whom would most likely benefit in terms of dietary management of Alzheimer's AMD, mild cognitive impairment, Parkinson's disease, complications of diabetes, including dibateic neuropathy and diabetic retinopathy where serum “gly-cocopper” and zinc deficiency are highly noted. The inventors are not aware of any oral zinc dose greater than 100 mg elemental zinc as ever having been tested or available. Accordingly, it is an object of the present invention to describe an oral dosage form of zinc containing over 100 mg of elemental zinc. More particularly, a dosage form containing over 100 mg elemental zinc that achieves zero or a low rate of gastric side effects.

The gastroretentive/sustained release zinc/cysteine tablets of the present invention achieve their GR through floatation which is achieved through a combination of effervescence (potassium bicarbonate and citric acid) and swelling (carbopol). This is just one example that may be used to achieve the same effect.

As an effervescent, these tablets are subject to premature effervescence if exposed to moisture. Such reaction releases CO₂ gas.

We have embarked on a 4-6 week study taking these tablets daily to see if they can affect free copper and/or ceruloplasmin at 4 or 6 weeks.

Premature effervescence can occur with these formulations.

Like other effervescent products such as Alka-Seltzer, this can most likely be overcome by individual packaging. Alka-Seltzer also uses heat treated sodium bicarbonate which is evident on the product ingredients label “sodium bicarbonate (heat treated)”.

It is apparently common to heat treat sodium bicarbonate prior to tableting to convert 2% to 10% of the bicarbonate to carbonate, creating a protective shell around each bicarbonate granule ad achieving an equilibrium that resists premature effervescence.

A pre-heat treated commercial product is available for sodium bicarbonate but the inventors can find none for potassium bicarbonate.

One of the inventors recently heat treated 25 tablets at 140 degrees F. for one hour and sealed them in a bottle with a desiccant and also took 25 non-heat treated tablets and placed them in a similar bottle.

After 3 hours, that inventor opened each bottle and observed no pressure in the bottle with heat-treated tablets while the bottle containing non-heat-treated tablets released a noticeable gas.

One can likely reduce the potential for premature effervescence by eliminating the citric acid from the formula. It is there to react with the bicarbonate and release CO₂. Since acid is present in the stomach, this reaction will occur to some extent, but not at the speed at which the current formulation accomplishes. Recently, the present inventors made some non-citric acid containing tablets and they failed to float until 20 minutes, which is most likely unacceptable since by that time they might have passed with the water with they were consumed into the intestines, thereby eliminating the highly important GR component of the present invention.

The present inventors believe that the immediate effervescent nature of the tablets is a major contributor to the lack of nausea experienced when the tablets of the present invention are consumed. It is believed that the release of gas causes the tablet to become mobile in the stomach, thereby reducing the local stomach wall irritation that George Brewer believes to be responsible for the nausea associated with IR zinc acetate and IR zinc sulphate capsules.

The present inventors are unsure whether eliminating the citric acid will completely eliminate the need to consider moisture-resistant packaging. While stability can likely be achieved with desiccated bottles, stress testing intended to resemble the bottle once opened and exposed to the high humidity environment of South Florida or any bathroom, will likely fail (regardless of the content of citric acid).

This leads to a product packaging solution that involves a low number of individual units per package. For example, bottles containing a 10-day supply or individual blister or pouch packaging (such as Alka-Seltzer with 2 per pouch).

For moisture control and to prevent or reduce CO₂ generation, a multi-tablet, sealed blister pack with reverse side punch out feature might work well where each tablet has its own contained area. Many antibiotics and some OTC antacids, and many other pill type products are packaged this way.

The present inventors have found two ways to improve the stability of potassium bicarbonate.

1. The first is to heat treat it to form a potassium carbonate shell (comprising for example 2% to 10% of material by weight) by the following reaction.
Potassium bicarbonate: KHCO₃
This is the equation which shows that when it is heated to between 100° C. and 120° C. it will decompose into K₂CO₃ (potassium carbonate), H₂O (water), and CO₂ (carbon dioxide).

2KHCO₃=>K₂CO₃+CO₂+H₂O

While heat treated sodium bicarbonate is commercially available, heat treated potassium bicarbonate is apparently not. The crystals are described in claim 1 of the U.S. Pat. No. 5,552,084 relating to aspirin, but how this is done is apparently not described.

The present inventors do not believe that a vacuum oven is necessary for this process nor that 100° C. to 120° C. will be necessary. Using a simple convection oven at 140° F. (60° C.) for 1 hour seems to work fine.

In process controls and testing of various heating parameters can be accomplished by weighing the starting material (before heating) and after heating (the release of CO₂ will indicate amount of conversion and reduce the weight (provided water is not accumulated from the air which should not be a problem even in a convection oven, provided the oven is pre-heated).

The stability of the potassium bicarbonate can then be tested after heating at various temperatures by leaving it in a high RH (relative humidity) environment and sequentially weighing it to observe changes in weight and comparing the various tests.

The present inventors still want the material to be reactive and release CO₂ in water, so this can measured by adding water to it or it to water and measuring pressure for example.

Ultimately, tablets should be made to see if they still readily float in the time frame the inventors prefer (under 1 minute).

2. The second process is to coat the potassium bicarbonate crystals by mixing them and thus coating them with a small percentage of zinc oxide.

This process is apparently patented by the makers of Arm & Hammer utilizing magnesium oxide, but does not claim zinc oxide although it describes zinc oxide as an example.

This process and the process of how to test the material is described in U.S. Pat. No. 5,552,084 and uses a sifting technique since unstable potassium bicarbonate will have a tendency to aggregate.

The present invention includes a formulation of oral effervescent GR/IR zinc acetate for Wilson's disease with the benefit of reduced nausea etc.

All measurements disclosed herein are at standard temperature and pressure, at sea level on Earth, unless indicated otherwise. All materials used or intended to be used in a human being are biocompatible, unless indicated otherwise.

The foregoing embodiments are presented by way of example only; the scope of the present invention is to be limited only by the following claims.

1. An oral dosage form of zinc for human consumption containing potassium bicarbonate and over 100 mg of elemental zinc.

2. An oral dosage form of zinc for human consumption containing potassium bicarbonate as an antacid.

3. An oral dosage form of zinc for human oral consumption that contains an effervescent agent, other than a lozenge or mouthwash.

4. An oral dosage form of zinc that provides over 50 mg of elemental zinc in the first hour and at least 50 mg over at least 6 hours.

5. An oral dosage form of zinc containing at least 50 mg of elemental zinc and a non-cellulose swelling agent.

6. An oral dosage form of zinc that approximates the bioavailability curve described in FIG. 2.

7. An oral dosage form of zinc containing potassium bicarbonate and at least 50 mg of elemental zinc and cysteine.

8. A gastrorententive formulation containing potassium bicarbonate and zinc acetate.

9. A gastrorententive formulation containing zinc carnosine.

10. A composition including the ingredients listed in Rows 1-4 of Column 1 or Column 2 of Table 1 in the specification.

11-13. (canceled)

14. The composition of claim 10, including the ingredients listed in Rows 1-4 of Column 1.

15. The composition of claim 10, including the ingredients listed in Rows 1-5.

16. The composition of claim 10, including the ingredients listed in Rows 1-6.

17. The composition of claim 10, including the ingredients listed in Rows 1-4 and 6.

18. A tablet made with the composition of claim 10.

19. The invention of claim 16, wherein the effervescence agent is heat treated.

20. Multiple tablets of claim 18, packaged in quantities of fewer than 10 tablets.

21. (canceled)

22. A method of providing zinc to a subject in need of treatment for deficiency of zinc comprising administering to the subject an effective amount of the composition of claim 10.

23. A method of providing zinc to a subject in need of treatment for deficiency of zinc comprising administering daily to the subject a tablet of claim 18.

24. A method of providing zinc to a subject in need of treatment for deficiency of zinc comprising administering twice daily to the subject a tablet of claim 18.

25. The method of claim 22, wherein the composition or tablet is administered orally.

26. The method of claim 22, wherein the amount administered daily is 25-300 mg bioavailable zinc.

27. (canceled)

28. The method of claim 22, wherein composition is administered for six months.

29. A pharmaceutical composition suitable for administration to a human containing two or more forms of zinc.

30. The pharmaceutical composition of claim 29, wherein at least one of the forms is a zinc salt.

31. The pharmaceutical composition of claim 29, wherein each of the forms has different solubility characteristics.

32. The pharmaceutical composition of claim 29, containing at least one free zinc salt and zinc bound to proteins or amino acids.

33. A pharmaceutical composition suitable for administration to a human containing two or more zinc salts.

34. The pharmaceutical composition of claim 33, wherein each of the salts has different solubility characteristics.

35. The pharmaceutical composition of claim 29 that are immediate release, delayed release, gastroretentive and/or sustained release.

36. The pharmaceutical composition of claim 29 that are effervescent.

37. (canceled)

38. A pharmaceutical composition suitable for administration to a human containing a calcium channel blocker plus zinc.

39. A pharmaceutical composition suitable for administration to a human containing potassium bicarbonate as an antacid and zinc.

40. A pharmaceutical composition suitable for administration to a human containing potassium and zinc.

41-42. (canceled)

43. A pharmaceutical composition suitable for administration to a human containing distilled water, zinc, and potassium.

44. (canceled)
